(19) United States
(12) Patent Application Publication
(10) Pub. No.: US 2008/0243047 A1
(43) Pub. Date: Oct. 2, 2008

(54) ULTRASOUND WOUND CARE DEVICE

(76) Inventor: Eilaz P. Babaev, Minnetonka, MN (US)

Correspondence Address: Bacoustics, LLC, 5929 BAKER ROAD, SUITE 470, MINNETONKA, MN, 55345, US

(21) Appl. No.: 11/691,969

(22) Filed: Mar. 27, 2007

(51) Int. Cl. A61M 37/00

(52) U.S. Cl. 604/22; 601/2

(57) ABSTRACT

The present invention relates to a method of treating wounds by applying ultrasonic energy to a wound to inactivate, destroy, and/or remove infectious agents, and/or deliver a coupling medium to debride, cleanse, and/or sterilize the wound. The device of the present invention comprises an ultrasound generator, an ultrasound transducer, a transducer tip at the distal end of the ultrasound transducer, and a radiation surface tilted with respect to the longitudinal axis of the transducer tip. Ultrasonic waves emanating from the tilted radiation surface of the present invention may travel through the air directly to the wound, and/or may be applied to the wound through a coupling medium.

### BACKGROUND OF THE INVENTION

The present invention relates to a wound care device, more particularly, a wound care device utilizing ultrasound energy for effective cleansing, sterilizing, debriding, inactivating, destroying, and/or removing infectious agents and/or other contaminants that may be present in a wound.

Wounds encountered in clinical practice can be hard to treat, slow to heal, and difficult to manage. The pain produced by such wounds disables the patient. An unhealed wound's susceptibility to infection increases a patient's morbidity and mortality. These wounds cause patients to experience severe emotional and physical distress as well as creating a significant financial burden on the patients and the healthcare system.

A wound cannot be properly diagnosed until all foreign materials, necrotic tissues, and/or infected tissues are removed. Damaged tissue, necrotic tissue and/or infected tissue must be removed in order to improve the healing potential of the remaining healthy tissue. This removal process is known as debridement. In general, debridement can either be done surgically, mechanically, chemically, and/or with maggot therapy, and these procedures are well-known in the art. These procedures can be tedious and can lead to the accidental removal of healthy tissue. Additionally, these procedures, especially surgical debridement can lead to further possible complications such as, but not limited to, bleeding, infection and delayed healing. The patient experiences great discomfort and pain from the procedures for treating the wound. These procedures fail to sufficiently clean the wound, disinfect the wound, inactivate and/or remove bacteria cells and/or foreign organisms that may be present in the wound.

Using ultrasound energy for treating wounds is well-known in the art. Ultrasonic energy is applied to a wound surface by direct contact or indirectly through a coupling medium. Examples of such devices can be found in U.S. Pat. No. 6,478,754 to Babaev; U.S. Pat. No. 6,533,803 to Babaev; U.S. Pat. No. 6,569,099 to Babaev; U.S. Pat. No. 6,663,554 to Babaev; U.S. Pat. No. 6,761,729 to Babaev; U.S. Pat. No. 6,916,296 to Soring et al.; U.S. Pat. No. 6,960,173 to Babaev, U.S. Pat. No. 6,964,647 to Babaev; U.S. Pat. No. 7,025,735 to Soring et al; and WIPO Patent WO 1997/017933 to Babaev.

These ultrasonic devices transmit ultrasonic energy to the wound usually through a radiation surface located at the distal end of either a transducer tip or horn. The radiation surfaces of these devices are usually flat or rectangular in shape, and emit ultrasonic waves in a manner parallel to the radiation surfaces' central axis. A coupling medium may carry ultrasonic energy to the wound when applied to the wound. However, the design of the radiation surface of these devices is not without problems: the emitted ultrasonic waves fail to aggressively debride the wound and remove effectively necrotic and/or infected tissues.

Since debridement of the wound is crucial to the healing potential of the wound, there is a need for a wound care device that effectively and adequately removes necrotic tissues, damaged tissues and/or infected tissues, supplies drugs to the wound, and/or inactivates, and/or destroys infectious agents that may be present in the wound.

### BRIEF SUMMARY OF THE INVENTION

The present invention is directed towards a device for the treatment of wounds. The device applies ultrasound energy to a wound surface to inactivate and/or destroy infectious agents that may be present in a wound, and/or delivers a coupling medium to debride, cleanse, and/or sterilize a wound. The device of the present invention comprises an ultrasound generator, an ultrasound transducer, a transducer tip at the distal end of the ultrasound transducer, and a radiation surface tilted with respect to the longitudinal axis of the transducer tip. Ultrasonic waves emanating from the tilted radiation surface of the present invention may travel through the air to the wound, and/or may be applied to the wound through a coupling medium.

The tilted radiation surface is placed in front of the wound, in close proximity to the wound surface. The ultrasound transduce is activated. Ultrasound energy generated by the transducer is then transmitted to the transducer tip. Activating the transducer generates ultrasound energy that is emitted from the tilted radiation surface as ultrasonic waves. Ultrasonic waves emanating from the tilted radiation surface may create the near field. Ozone is created in the near field and may be delivered to the wound. Ozone may enter the surface of the wound and penetrate beneath the wound surface. Ozone may be delivered to the wound through a variety of ways such as but not limited to, delivery by the ultrasonic waves emanating from the present invention into the wound, dissolving the ozone in the coupling medium, and/or by diffusion. The ozone inactivates and/or destroys any foreign organisms and/or materials within and/or beneath the surface of the wound. Ultrasonic energy may be delivered to the wound using a coupling medium such as, but not limited to saline, gels, and/or medications. The coupling medium may be used to wash, cleanse and/or sterilize the wound surface. Coupling medium may wash away and/or remove inactivated foreign organisms, materials, and/or bacterial cells.

The present invention is different from prior art wound care devices utilizing the “Babaev effect” to produce a spray; an example is the U.S. Pat. No. 6,569,099 to Babaev. This prior art device delivers liquids to the lateral surface of an ultrasound transducer tip whereby the liquid is pulled to the radiation surface by a negative pressure created by the atomization of liquid at the radiation surface by the ultrasonic waves emanating from the radiation surface. However, the device of the present invention does not utilize the Babaev effect to deliver a liquid coupling medium to the radiation surface.

Coupling medium may be delivered onto the tilted radiation surface by dropping the coupling medium from an elevated position directly above the tilted radiation surface. Similarly, coupling medium may also be delivered to the tilted radiation surface through one or more channels terminating at an orifice within tilted radiation surface. The coupling medium may be delivered to the tilted radiation surface from such a channel by applying force or pressure to the coupling medium.

Ultrasonic waves emanating from the tilted radiation surface may also be applied to the wound surface for a period of time, such as from one second to as much as a minute or more, depending on the wound. A coupling medium carrying ultrasonic energy may also be delivered to the wound. Ultrasonic energy, and/or a coupling medium may be delivered to the wound simultaneously. When sonicated liquids are used as the coupling medium, liquids delivered to the wound irrigates the wound, removing devitalized tissues, necrotic tissues, infected tissues, foreign materials, and/or other contaminants which may impede the healing process of wounds.

The device of the present invention is particularly advantageous on a wound surface and surrounding tissues because directing ultrasound energy to the wound and/or applying sonicated liquids to a wound increases blood flow, disinfects wounds, and/or improves overall healing time of wounds. Another advantage of the present invention is the effective debridement of the wound, which is critical to the healing progress of the wound.

A flat and/or rectangular shaped radiation surface makes it difficult for ultrasonic waves to access wounds, especially narrow wounds, and effectively debride the wound, because only a portion of ultrasonic waves propagated in a manner parallel to the radiation surfaces' central axis reaches the wound surface. Coupling mediums such as, liquids, medications and/or saline introduced to the flat radiation surface fall off the radiation surface without reaching the wound, thus leading to waste of the sonicated coupling medium and/or liquids. Furthermore, since only a portion of the sonicated coupling medium reaches the wound, the wound is not adequately debrided, cleaned and/or sterilized.

One of the major advantages of the device of the present invention is the effective treatment of narrow wounds. Generally, narrow wounds can be very hard to treat for failure of having access to such wounds. Thus, debridement of such wounds cannot be effectively done due to lack of access of the ultrasonic waves to the wounds. The tilted radiation surface of the present invention delivers ultrasonic waves and/or coupling medium to the wound by propagating ultrasonic waves in a manner that is angled with respect to the longitudinal axis of the tip, thereby providing the sonicated coupling medium and/or ultrasonic waves access to narrow wound beds. Delivering ultrasonic waves to a wound along a vector with both a vertical and horizontal component with respect to the wound, the present invention provides an ultrasonic scrapping action, thereby removing dead tissues, damaged tissues, infected tissues and/or foreign materials that may be present in the wound. Thus, the tilted radiation surface provides aggressive and effective debridement of narrow wound beds.

One aspect of the device and method of the present invention may be to treat wounds and assist in the healing process of wounds.

Another aspect of the device and method of the present invention may be to effectively remove necrotic tissues, damaged tissues, infected tissues, and/or other contaminants from the wound.

Another aspect of the device and method of the present invention may be to deliver coupling medium to the wound.

Another aspect of the device and method of the present invention may be to sterilize the wounds.

Another aspect of the device and method of the present invention may be to treat narrow wound beds.

Another aspect of the device and method of the present invention may be to inactivate and/or destroy infectious agents that may be present in the wound.

Another aspect of the device and method of the present invention may be to increase blood flow to the wound bed.

These and other aspects of the invention will become more apparent from the written description and figures below.

### BRIEF DESCRIPTION OF THE SEVERAL VIEWS OF THE DRAWING

The present invention will be shown and described with reference to the drawing of preferred embodiments and will be clearly understood in details.

FIG. 1 depicts a cross-sectional view of the tilted radiation surface located at the distal end of the transducer tip.

FIG. 2 depicts a cross-sectional view of the preset invention as liquid is delivered to the tilted radiation surface.

### DETAILED DESCRIPTION OF THE INVENTION

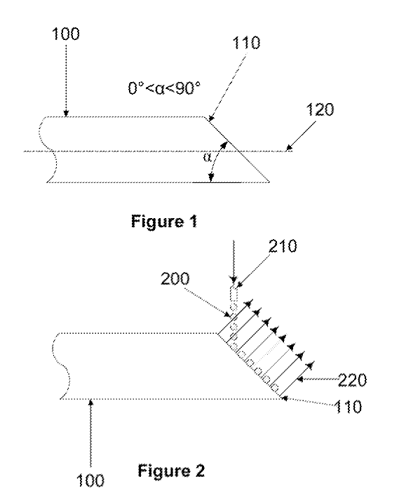

FIG. 1 depicts a cross-sectional view of the tilted radiation surface 110 located at the distal end of the transducer tip 100. The device of the present invention combines ultrasonic energy and coupling medium to treat wounds by debriding, sterilizing, destroying, inactivating infectious organisms, and/or removing foreign materials and/or other contaminants that may be present in a wound. The device of the present invention comprises an ultrasound generator (not shown), ultrasound hank (not shown), transducer tip 110 at the distal end of ultrasound transducer, and a radiation surface 110 tilted with respect to the longitudinal axis of the transducer tip 100. The tilted radiation surface 110 is at an angle α, so that α is in the range of 0<α<90° with respect to the longitudinal axis of the transducer tip 100. The tilted radiation surface 110 is designed in such a way that the spray is never parallel to the center axis 120.

FIG. 2 depicts a cross-sectional view of the present invention. The present invention may be used to treat wounds by placing a wound surface in close proximity to the tilted radiation surface 110. As the ultrasound transducer (not shown) is activated, ultrasound energy is carried through transducer tip 100 to tilted radiation surface 110. Ultrasound energy passing through the transducer tip 100 is emitted from the tilted radiation surface 110, generating a near field. According to the theory of classical physics, free electrons are electrons not held in molecular orbit. Negative ions are free electrons. Positive ions are molecules that have lost electrons and are polarized. It is important to notice that significant ultrasonic air ionization process occurs more durable and active in-between the ultrasound emitting surface and a barrier in front of it, such as a wound surface in this present invention. In this condition, ionization of air occurs in the near field-far field interface between the ultrasound emitting surface and the barrier during sonication period.

The length, L, of the near field (Fresnel Zone) is equal to L=r²/λ=d²/4λ, where r is the radius and d is the diameter of the ultrasound emitting surface or distal end diameter of ultrasonic tip, and λ is the ultrasound wavelength in the medium of propagation. Maximum ultrasound intensity occurs at the interface between the near field (Fresnel zone) and the far field (Fraunhofer zone). Beam divergence in the far field results in a continuous loss of ultrasound intensity with distance from the transducer. As the transducer frequency is increased, the wavelength λ decreases, thus resulting in an increase in the length of the near field. Ionization time can be from fraction of seconds up to minutes depending on ultrasound energy parameters and design of the ultrasound transducer or tip.

Emitted ultrasonic waves 220 create ozone. To clarify and describe the ultrasound air ionization effect, stable air (mainly nitrogen and oxygen) molecules are not polarized, and an ultrasound field does not affect them. Air also contains many free electrons (negative ions) which move back and forth in the ultrasound field. Overstressing of air (preferably between ultrasound emitting surface and barrier) at greater than about 1 w/cm² [watts per square centimeter] can cause the free electrons from stable molecules in the air to attain sufficient energy to knock the free electrons from stable molecules in the air. These newly freed electrons knock off even more electrons, producing more negative and positive ions. When the oxygen molecules in the air lose electrons they become polarized positive ions. These positive ions form ozone:

O₂→O+O

O+O₂→O₃

The fast-moving negative ions, as well as the slower heavy positive ions, bombard the wound surface when applied to the wound, eventually destroying and/or inactivating foreign organisms, bacterial cells and/or other contaminants that may be present in a wound.

In the pictured embodiment, coupling medium 200 such as, liquid medicines and/or saline may be delivered from an elevated position above tilted radiation surface 110 through a valve 210. Coupling medium may be used to wash, cleanse and/or sterilize the wound after ozone is applied to the wound. Coupling medium 200 is sonicated as it comes in contact with the ultrasonic waves emanating from the tilted radiation surface 110. Sonicated coupling medium 200, may be delivered to the wound from tilted radiation surface 110. Coupling medium 200 may penetrate the wound and may be delivered beneath the wound surface. Ultrasonic waves 220 represent the direction in which sonicated coupling medium 200 is propagated. Sonicated coupling medium 200 flushes the wound and penetrates beneath the wound surface, removing dead tissues, damaged tissues, and/or infected tissues and/or any infectious agents that may be present in the wound. Additionally, sonicated coupling medium 200 may provide therapeutic benefits to the wound by sterilizing the wound, increasing blood flow to the wound, and/or increasing healing time of the wound.

The sonicated liquids delivered to the wound surface may debride the wound, wash, clean the wound, and/or sterilize the wound. Coupling medium 200 may be delivered onto the tilted radiation surface 110 by dropping the coupling medium 200 from an elevated position directly above the tilted radiation surface 110. Similarly, coupling medium 200 may also be delivered to the tilted radiation surface 110 through one or more channels terminating at an orifice within tilted radiation surface 110. The coupling medium 200 may be delivered to the tilted radiation surface 110 from such a channel by applying force or pressure to the coupling medium 200.

Ultrasonic waves 220 emanating from the tilted radiation surface 110 may also be applied to the wound. Ultrasonic waves 220 may travel through the air to the wound surface and may be applied to the wound for a prespecified period of time such as from fraction of a second to as much as a minute or more, depending on the factors and/or circumstances surrounding the wound. It is preferable that the tilted radiation surface does not come in contact with the wound. Coupling medium may also be introduced to the wound at this time, or at a later time, as needed. Ultrasonic waves 220 may allow the coupling medium 200 to penetrate the surface of the wound. The duration of the wound treatment depends on the type of wound and the factors and/or circumstances surrounding the wound. These factors and/or circumstances may include the type of tissue being treated, the condition of the wound, size of the wound, and/or location of the wound. Ultrasonic waves 220 travel in the direction depicted by the arrows in FIG. 2. The direction of travel of ultrasonic waves 220 is created due to the tilted radiation surface 110 angle α.

The tilted radiation surface 110 provides emanating ultrasonic waves 220 access to narrow wounds. Emitted ultrasonic waves 220 are propagated in a manner that is angled with respect to the longitudinal axis of the transducer tip 100. Ultrasonic waves 220 applied to the wound surface directly and/or through a coupling medium 200 debride the wound by removing necrotic tissues, and/or infected tissues, damaged tissues, and/or any infectious agents as ultrasonic waves 220 comes in contact with the narrow wound bed.

Although specific embodiments have been illustrated and described herein, it will be appreciated by those of ordinary skill in the art that any arrangement that is calculated to achieve the same purpose may be substituted for the specific embodiments shown. It is to be understood that the above description is intended to be illustrative and not restrictive. Combinations of the above embodiments and other embodiments will be apparent to those having skill in the art upon review of the present disclosure. The scope of the present invention should be determined with reference to the appended claims, along with the full scope of equivalents to which such claims are entitled.

1. (canceled)

2. (canceled)

3. An ultrasound wound care device comprising:
an ultrasound generator driving;
an ultrasound transducer having a distal end;
a transducer tip at the distal end, the transducer tip having a longitudinal axis;
a radiation surface at the transducer tip distal end;
the radiation surface emitting ultrasound energy at an intensity sufficient for producing ozone; and
the radiation surface being tilted at an angle between 0 degrees and 90 degrees to the longitudinal axis to direct the ozone.

4. The device of claim 3 having an interface between a near field and a far field with a wound surface located near the interface.

5. The device of claim 3 having the ozone delivered to a wound surface.

6. The device of claim 3 providing a coupling medium to the radiation surface, the coupling medium being directed toward a wound surface.

7. The device of claim 3 wherein the ultrasound energy provides ionization of air molecules.

8. The device of claim 3 wherein a coupling medium carries medications to a wound surface.

9. The device of claim 3 wherein a coupling medium debrides a wound surface.

10. The device of claim 3 wherein a coupling medium washes a wound surface.

11. The device of claim 3 wherein a coupling medium sterilizes a wound surface.
